(19) United States
(12) Patent Application Publication
(10) Pub. No.: US 2013/0142882 A1
(43) Pub. Date: Jun. 6, 2013

(54) METHODS AND COMPOSITIONS FOR TREATMENT, MODIFICATION AND MANAGEMENT OF BONE CANCER PAIN

(71) Applicant: GOLDEN BIOTECHNOLOGY CORPORATION, New Taipei City (TW)

(72) Inventors: Sheng-Yung Liu, New Taipei City (TW); San-Bao Hwang, New Taipei City (TW); Wu-Che Wen, New Taipei City (TW)

(73) Assignee: GOLDEN BIOTECHNOLOGY CORPORATION, New Taipei City (TW)

(21) Appl. No.: 13/677,217

(22) Filed: Nov. 14, 2012

Related U.S. Application Data
(60) Provisional application No. 61/560,185, filed on Nov. 15, 2011.

(51) Int. Cl. A61K 31/122; A61K 45/06; A61K 31/19

(52) U.S. Cl. CPC A61K 31/122; A61K 31/19; A61K 45/06
USPC 424/600; 514/690; 514/557; 514/171; 514/163; 514/164; 514/282; 514/217; 514/356; 514/329

(57) ABSTRACT

The present invention provides methods and compositions for treating, preventing, modifying (reducing), or managing bone cancer pain by cyclohexenone compounds.

### CROSS REFERENCE

This application claims the benefit of U.S. provisional application Ser. No. 61/560,185, filed Nov. 15, 2011, which is incorporated by reference in its entirety.

### BACKGROUND OF THE INVENTION

Bone cancer pain may arise in humans from either primary bone tumors or more commonly from bone metastases (such as from breast, prostate, and lung carcinomas). See Luger et al., Pain 99:397-406 (2002). This type of pain is difficult to treat due to its intermittent, progressive nature and its aggravation by movement. The predominant symptom in this model of pain is mechanical allodynia. Thermal hyperalgesia and mechanical hyperalgesia has also been demonstrated as measured by the weight bearing difference in the two hind limbs (Medhurst et al., 2002). Treatment of bone pain, especially bone cancer pain, in human patients is largely limited to the use of opioids, however the efficacy of potent opioids is minimal, and effective doses produce a range of debilitating side effects.

### SUMMARY OF THE INVENTION

In one aspect provides herein treating, reducing, or managing bone cancer pain comprising administering to a subject a therapeutically effective amount of a compound having the structure:

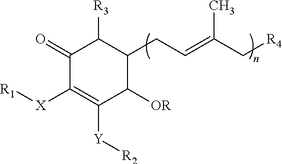

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

### Incorporation by Reference

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-B show illustrative effective results of an exemplary Compound 1 on mechanical allodynia in a model of bone cancer pain. Data are expressed as mean ±s.e. mean. *P<0.05, **P<0.01 and ***P<0.001 when compared to vehicle (ANOVA and Dunnett's test). #P<0.05, ##P<0.01 and ###P<0.001 when compared to vehicle (Kruskall Wallis and Dunn's test).^{$}P<0.05, ^{$$}P<0.01 and ^{$$$}P<0.001 when compared to vehicle (unpaired, Student's t test). ^{†††}P<0.001 when compared to vehicle (Mann Whitney U-test).

FIG. 2A-B show illustrative effective results of an exemplary Compound 1 on the development of mechanical allodynia (Day 6 PO) following twice daily from the day of surgery. Data are expressed as mean±s.e. mean. #P<0.05 when compared to vehicle (Kruskall Wallis and Dunn's test). ^{$}P<0.05 when compared to vehicle (unpaired, Student's t-test).

FIG. 3A-B show illustrative effective results of an exemplary Compound 1 on the development of mechanical allodynia (Day 12 PO) following twice daily from the day of surgery. Data are expressed as mean±s.e. mean. **P<0.01 and ***P<0.001 when compared to vehicle (ANOVA and Dunnett's test). ^{$$}P<0.01 and ^{$$$}P<0.001 when compared to vehicle (unpaired, Student's t-test).

FIG. 4A-B show illustrative effective results of an exemplary Compound 1 on the development of mechanical allodynia (Day 14 PO) following twice daily from the day of surgery. Data are expressed as mean±s.e. mean. #P<0.05 and ###P<0.001 when compared to vehicle (Kruskall Wallis and Dunn's test). ^{$$$}P<0.001 when compared to vehicle (unpaired, Student's t-test).

FIG. 5A-B show illustrative effective results of an exemplary Compound 1 on the development of mechanical allodynia (Day 19 PO) following twice daily from the day of surgery. Data are expressed as mean±s.e. mean. #P<0.05, ##P<0.01 and ###P<0.001 when compared to vehicle (Kruskall Wallis and Dunn's test). ^{$$$}P<0.001 when compared to vehicle (unpaired, Student's t-test).

FIG. 6A-B show illustrative effective results of an exemplary Compound 1 on the development of mechanical allodynia (Day 21 PO) following twice daily from the day of surgery. Data are expressed as mean±s.e. mean. *P<0.05 and ***P<0.001 when compared to vehicle (ANOVA and Dunnett's test). #P<0.05 and ###P<0.001 when compared to vehicle (Kruskall Wallis and Dunn's test). ^{$$$}P<0.001 when compared to vehicle (unpaired, Student's t-test). ^{†††}P<0.001 when compared to vehicle (Mann Whitney U-test).

### DETAILED DESCRIPTION OF THE INVENTION

Common treatments for bone cancer pain in human patients are largely limited to the use of opioids. However, the efficacy of potent opioids is minimal, and effective doses produce a range of debilitating side effects. The invention cyclohexenone compounds, in some embodiments, are obtained from extracts of natural products and provide reduced complications and/or side effects. In some embodiments, provided herein are methods for treating, preventing, modifying (reducing), or managing bone cancer pain by administering a cyclohexenone compound provided herein to a subject (e.g. a human). The cyclohexenone compounds provide therapeutic benefit to a subject being treated for bone cancer pain (see Examples 1-3).

In some embodiments, there are provided methods for treating, preventing, reducing or managing bone cancer pain comprising administering to a subject a therapeutically effective amount of a compound having the structure:

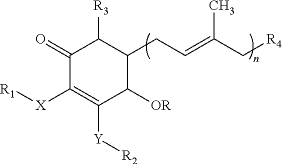

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃; R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

Bone is one of the most common locations for metastasis. While any type of cancer is capable of forming metastatic tumors within bone, the microenvironment of the marrow tends to favor particular types of cancer, including prostate, breast, and lung cancers. Particularly in prostate cancer, bone metastases tend to be the only site of metastasis.

In some embodiments, the bone cancer pain is from cancer originated in bone. In some embodiments, the bone cancer pain is from osteosarcoma. In some embodiments, the bone cancer pain is from cancer metastasized to bone. In certain embodiments, the bone cancer pain is from breast cancer, prostate cancer, lung cancer, renal cancer, liver cancer, kidney cancer, bladder cancer, thyroid cancer, cervical cancer, colon cancer, or other similar cancer metastasized to bone. In certain embodiments, the bone cancer pain is from prostate cancer metastasized to bone. In certain embodiments, the bone cancer pain is from breast cancer metastasized to bone. In certain embodiments, the bone cancer pain is from lung cancer metastasized to bone. In certain embodiments, the bone cancer pain is from renal cancer metastasized to bone. In certain embodiments, the bone cancer pain is from esophageal cancer, or nasopharyngeal cancer metastasized to bone. In certain embodiments, the bone cancer pain is from sarcoma metastasized to bone. See Examples 1-3.

In some embodiments, the cyclohexenone compounds provided herein also show significant protective effects on the development of mechanical allodynia (Example 2).

In certain embodiments, there are provided methods for treating, preventing, reducing or managing mechanical allodynia comprising administering to a subject a therapeutically effective amount of a compound having the structure:

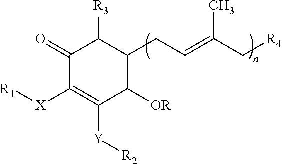

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)O R₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

In some embodiments, the cyclohexenone compound provided herein for treating, preventing, modifying (reducing), or managing bone cancer pain or mechanical allodynia having the structure

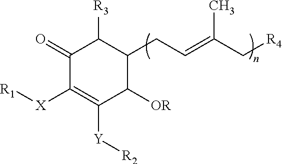

is prepared synthetically or semi-synthetically from any suitable starting material. In other embodiments, the cyclohexenone compound is prepared by fermentation, or the like. For example, Compound 1 (also known as Antroquinonol™ or “Antroq”) or Compound 3, in some instances, is prepared from 4-hydroxy-2,3-dimethoxy-6-methylcyclohexa-2,5-dienone. The non-limited exemplary compounds are illustrated below.

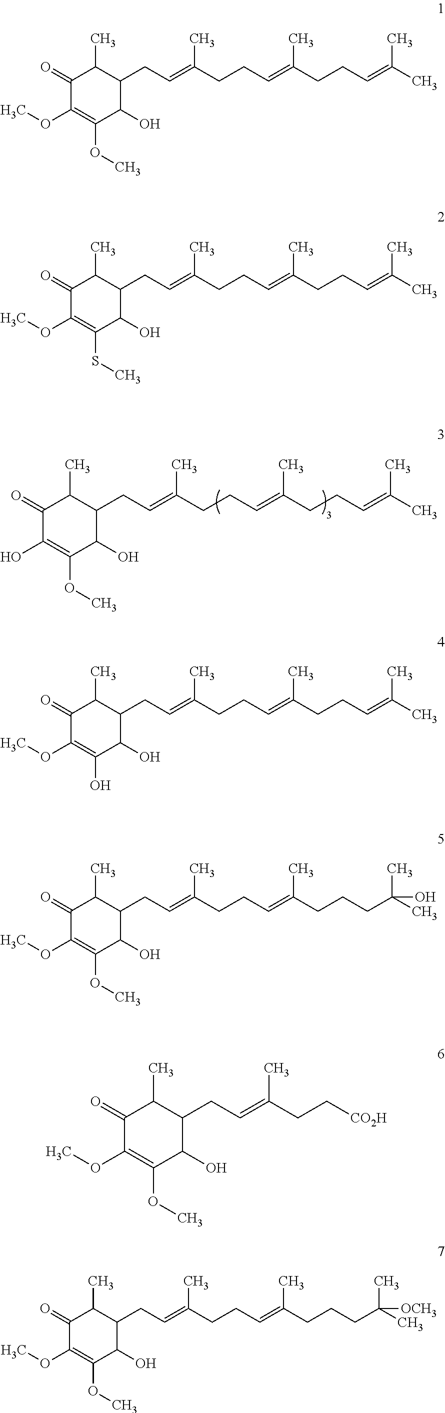

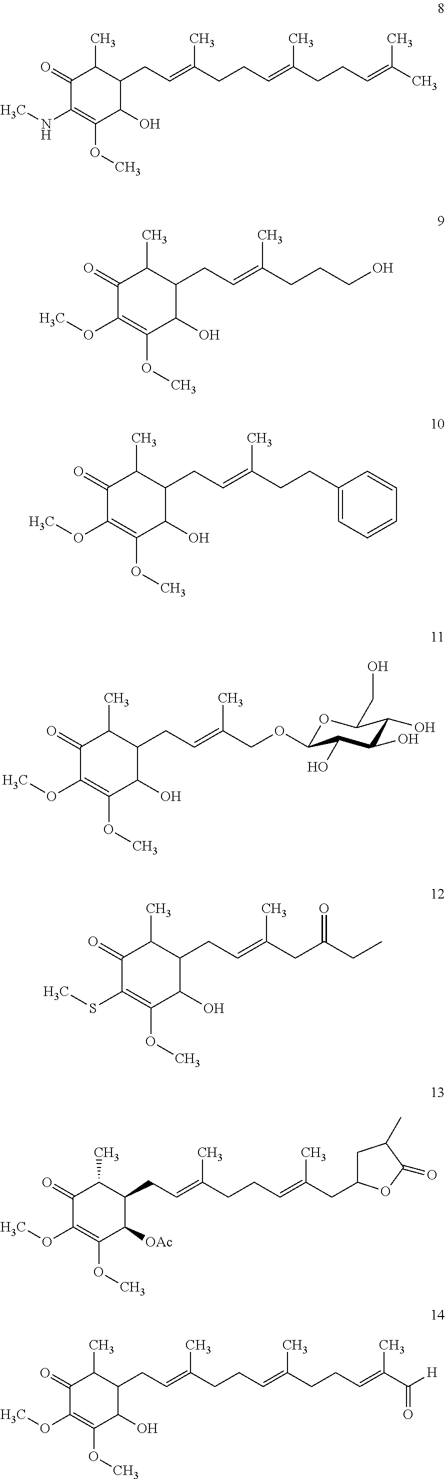

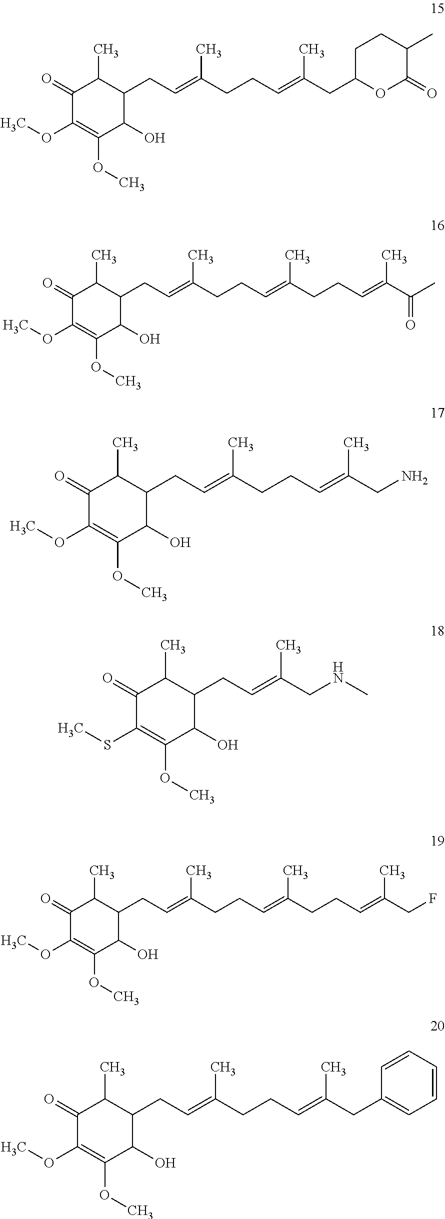

In other embodiments, the cyclohexenone compound provided herein for treating, preventing, modifying (reducing), or managing bone cancer pain or mechanical allodynia having the structure

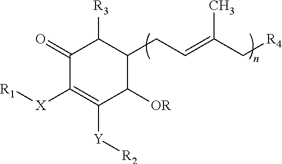

is isolated from the organic solvent extracts of Antrodia camphorate. In some embodiments, the organic solvent is selected from alcohols (e.g., methanol, ethanol, propanol, or the like), esters (e.g., methyl acetate, ethyl acetate, or the like), alkanes (e.g., pentane, hexane, heptane, or the like), halogenated alkanes (e.g., chloromethane, chloroethane, chloroform, methylene chloride, and the like), and the like. For example, exemplary Compounds 1-7 are isolated from organic solvent extracts. In certain embodiments, the organic solvent is alcohol. In certain embodiments, the alcohol is ethanol. In some embodiments, the cyclohexenone compound is isolated from the aqueous extracts ofAntrodia camphorate.

In some embodiments, R is a hydrogen, C(═O)C₃H₈, C(═O)C₂H₅, or C(═O)CH₃. In some embodiments, R₁ is a hydrogen or methyl. In certain embodiments, R₂ is a hydrogen, methyl, ethyl, propyl, butyl, pentyl or hexyl. In some embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In some embodiments, R₄ is C₂H₅C(CH₃)₂OH, C₂H₅C(CH₃)₂O CH₃, CH₂COOH, C₂H₅COOH, CH₂OH, C₂H₅OH, CH₂Ph, C₂H₅Ph, CH₂CH=C(CH₃)(CHO), CH₂CH═C(CH₃)(C(═O)CH₃), 5 or 6-membered lactone, C₂-C₈alkenyl, C₂-C₈alkynyl, aryl, and glucosyl, wherein the 5 or 6-membered lactone, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

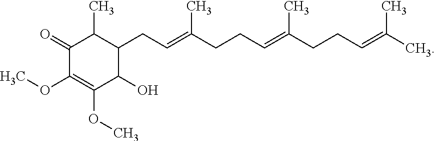 [SMILES: COC1=C(OC)C(O)C(C/C=C(\C)CC/C=C(\C)CCC=C(C)C)C(C)C1=O]

### Certain Pharmaceutical and Medical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group (which means that it does not contain any carbon-carbon double bonds or carbon-carbon triple bonds) or the alkyl group may be an unsaturated alkyl group (which means that it contains at least one carbon-carbon double bonds or carbon-carbon triple bond). The alkyl moiety, whether saturated or unsaturated, may be branched, or straight chain.

The “alkyl” group may have 1 to 12 carbon atoms (whenever it appears herein, a numerical range such as “1 to 12 refers to each integer in the given range; e.g., “1 to 12 carbon atoms” means that the alkyl group may consist of 1 carbon atom, 2 carbon atoms, 3 carbon atoms, etc., up to and including 12 carbon atoms, although the present definition also covers the occurrence of the term “alkyl” where no numerical range is designated). The alkyl group of the compounds described herein may be designated as “C₁-C₈ alkyl” or similar designations. By way of example only, “C₁-C₈ alkyl” indicates that there are one, two , three, four, five, six, seven or eight carbon atoms in the alkyl chain. In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₈ alkyl.

The term “alkylene” refers to a divalent alkyl radical. Any of the above mentioned monovalent alkyl groups may be an alkylene by abstraction of a second hydrogen atom from the alkyl. In one aspect, an alkylene is a C₁-C₁₂alkylene. In another aspect, an alkylene is a C₁-C₈alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like.

As used herein, the term “aryl” refers to an aromatic ring wherein each of the atoms forming the ring is a carbon atom. Aryl rings are formed by five, six, seven, eight, nine, or more than nine carbon atoms. Aryl groups are optionally substituted. In one aspect, an aryl is a phenyl or a naphthalenyl. In one aspect, an aryl is a phenyl. In one aspect, an aryl is a C₆-C₁₀aryl. Depending on the structure, an aryl group can be a monoradical or a diradical (i.e., an arylene group). In one aspect, an arylene is a C₆-C₁₀ arylene. Exemplary arylenes include, but are not limited to, phenyl-1,2-ene, phenyl-1,3-ene, and phenyl-1,4-ene.

The term “aromatic” refers to a planar ring having a delocalized it-electron system containing 4n+2π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “halo” or, alternatively, “halogen” or “halide” means fluoro, chloro, bromo or iodo.

The term “lactone” refers to a cyclic ester which can be seen as the condensation product of an alcohol group —OH and a carboxylic acid group —COOH in the same molecule. It is characterized by a closed ring consisting of two or more carbon atoms and a single oxygen atom, with a ketone group ═O in one of the carbons adjacent to the other oxygen.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles may be substituted with one or two oxo (═O) moieties, such as pyrrolidin-2-one.

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (i.e., an alkenylene group). In some embodiments, alkenyl groups are optionally substituted. Illustrative examples of alkenyl include, but are not limited to, ethenyl, 2-propenyl, 2-methyl-2-propenyl, 3-butenyl, 4-pentenyl, 5-hexenyl, 2-heptenyl, 2-methyl-l-heptenyl, and 3-cecenyl.

The term “alkynyl” as used herein, means a straight, branched chain, or cyclic (in which case, it would also be known as a “cycloalkenyl”) hydrocarbon containing from 2-10 carbons and containing at least one carbon-carbon triple bond formed by the removal of four hydrogens. In some embodiments, depending on the structure, an alkynyl group is a monoradical or a diradical (i.e., an alkynylene group). In some embodiments, alkynyl groups are optionally substituted. Illustrative examples of alkynyl include, but are not limited to, ethynyl, propynyl, butyryl, pentynyl, hexynyl, heptynyl, and the like.

The term “alkoxy” as used herein, means an alkyl group, as defined herein, appended to the parent molecular moiety through an oxygen atom. Illustrative examples of alkoxy include, but are not limited to, methoxy, ethoxy, propoxy, 2-propoxy, butoxy, tert-butoxy, pentyloxy, and hexyloxy.

The term “cycloalkyl” as used herein, means a monocyclic or polycyclic radical that contains only carbon and hydrogen, and includes those that are saturated, partially unsaturated, or fully unsaturated. Cycloalkyl groups include groups having from 3 to 10 ring atoms. Representative examples of cyclic include but are not limited to, the following moieties:

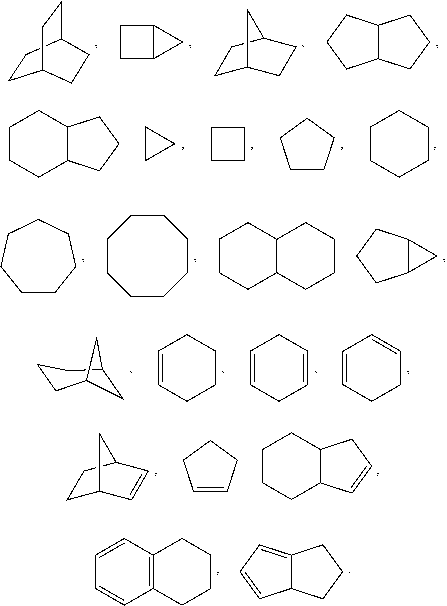

In some embodiments, depending on the structure, a cycloalkyl group is a monoradical or a diradical (e.g., a cycloalkylene group).

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “glucosyl” as used herein, include D- or L-form glucosyl groups, in which the glucosyl group is attached via any hydroxyl group on the glucose ring.

The term “acceptable” with respect to a formulation, composition or ingredient, as used herein, means having no persistent detrimental effect on the general health of the subject being treated.

Antrodia is a genus of fungi in the family Meripilaceae. Antrodia species have fruiting bodies that typically lie flat or spread out on the growing surface, with the hymenium exposed to the outside; the edges may be turned so as to form narrow brackets. Most species are found in temperate and boreal forests, and cause brown rot. Some of the species in this genus are have medicinal properties, and have been used in Taiwan as a Traditional medicine.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound (i.e., a cyclohexenone compound described herein) and a co-agent, are both administered to a patient simultaneously in the form of a single entity or dosage. The term “non-fixed combination” means that the active ingredients, e.g. a compound (i.e., a cyclohexenone compound described herein) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound (i.e., a cyclohexenone compound described herein) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing (reducing the risk of) additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Routes of Administration

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, the cyclohexenone compound, or a pharmaceutically acceptable salt, metabolite, solvate or prodrug thereof, is administered parenterally or intravenously. In other embodiments, the cyclohexenone compound, or a pharmaceutically acceptable salt, metabolite, solvate or prodrug thereof, is administered by injection. In some embodiments, the cyclohexenone compound, or a pharmaceutically acceptable salt, metabolite, solvate or prodrug thereof, is administered orally.

### Pharmaceutical Composition/Formulation

In some embodiments provide pharmaceutical compositions comprising a therapeutically effective amount of a compound having the structure:

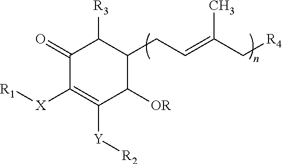

wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and n=1-12; or a pharmaceutically acceptable salt, metabolite, solvate or prodrug thereof; and a pharmaceutically acceptable excipient.

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound (i.e., a cyclohexenone compound described herein) and a pharmaceutically acceptable diluent(s), excipient(s), or carrier(s). In certain embodiments, the compounds described are administered as pharmaceutical compositions in which a compound (i.e., a cyclohexenone compound described herein) is mixed with other active ingredients, as in combination therapy. Encompassed herein are all combinations of actives set forth in the combination therapies section below and throughout this disclosure. In specific embodiments, the pharmaceutical compositions include one or more compounds (i.e., a cyclohexenone compound described herein).

A pharmaceutical composition, as used herein, refers to a mixture of a compound (i.e., a cyclohexenone compound described herein) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds (i.e., a cyclohexenone compound described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, a compound (i.e., a cyclohexenone compound described herein) is formulated in an aqueous solution. In specific embodiments, the aqueous solution is selected from, by way of example only, a physiologically compatible buffer, such as Hank's solution, Ringer's solution, or physiological saline buffer. In other embodiments, a compound (i.e., a cyclohexenone compound described herein) is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including a compound (i.e., a cyclohexenone compound described herein), are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of a compound (i.e., a cyclohexenone compound described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, the compounds (i.e., the cyclohexenone compounds described herein) are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound (i.e., a cyclohexenone compound described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of a compound (i.e., a cyclohexenone compound described herein) per 1 mL of solution. Each injector is capable of delivering only one dose of the compound.

In still other embodiments, the compounds (i.e., the cyclohexenone compounds described herein) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds (i.e., the cyclohexenone compounds described herein) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of a compound (i.e., a cyclohexenone compound described herein) is accomplished by means of iontophoretic patches and the like. In certain embodiments, transdermal patches provide controlled delivery of a compound (i.e., a cyclohexenone compound described herein). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound (i.e., a cyclohexenone compound described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds (i.e., cyclohexenone compounds described herein) are formulated for administration by inhalation. Various forms suitable for administration by inhalation include, but are not limited to, aerosols, mists or powders. Pharmaceutical compositions of a compound (i.e., a cyclohexenone compound described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include a compound (i.e., a cyclohexenone compound described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, the compounds (i.e., the cyclohexenone compounds described herein) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound (i.e., a cyclohexenone compound described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound (i.e., the cyclohexenone compounds described herein) described herein as an active ingredient. The active ingredient is in free-acid or free-base form, or in a pharmaceutically acceptable salt form. In addition, the methods and pharmaceutical compositions described herein include the use crystalline forms (also known as polymorphs), as well as active metabolites of these compounds having the same type of activity. All tautomers of the compounds described herein are included within the scope of the compounds presented herein. Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least one compound (i.e., the cyclohexenone compounds described herein) illustratively takes the form of a liquid where the agents are present in solution, in suspension or both. Typically when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound (i.e., a cyclohexenone compound described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (0 0.003% to about 0.02% w/v polysorbate 80, (g) 0.001% to about 0.05% w/v. polysorbate 20, (h) arginine, (i) heparin, (j) dextran sulfate, (k) cyclodextrins, (l) pentosan polysulfate and other heparinoids, (m) divalent cations such as magnesium and zinc; or (n) combinations thereof.

### Combination Treatments

In general, the compositions described herein and, in embodiments where combinational therapy is employed, other agents do not have to be administered in the same pharmaceutical composition, and in some embodiments, because of different physical and chemical characteristics, are administered by different routes. In some embodiments, the initial administration is made according to established protocols, and then, based upon the observed effects, the dosage, modes of administration and times of administration is modified by the skilled clinician.

In some embodiments, therapeutically-effective dosages vary when the drugs are used in treatment combinations. Combination treatment further includes periodic treatments that start and stop at various times to assist with the clinical management of the patient. For combination therapies described herein, dosages of the co-administered compounds vary depending on the type of co-drug employed, on the specific drug employed, on the disease, disorder, or condition being treated and so forth.

It is understood that in some embodiments, the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein.

Combinations of compounds (i.e., the cyclohexenone compound described herein) with other active agents that are capable of relieving or reducing pain are intended to be covered.

In some embodiments, the methods for treating, preventing (reducing the risk of), modifying (reducing), or managing bone cancer pain provided herein further comprise administering to the patient a therapeutically or prophylactically effective amount of at least one second active agent. In certain embodiments, the second active agent is capable of relieving or reducing pain. In some embodiments, examples of pain relieving or reducing agents include, but are not limited to, the following: an antidepressant, antihypertensive, anxiolytic, calcium channel blocker, muscle relaxant, non-narcotic analgesic, anti-inflammatory agent, cox-2 inhibitor, alpha-adrenergic receptor agonist, alpha-adrenergic receptor antagonist, ketamine, anesthetic, immunomodulatory agent, immunosuppressive agent, corticosteroid, hyperbaric oxygen, anticonvulsant, a combination thereof, or the like.

In some embodiments, the active agents are salicylic acid acetate, celecoxib, ketamine, gabapentin, carbamazepine, oxcarbazepine, phenytoin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, combinations thereof, or the like.

The combinations of the cyclohexenone compounds and pain relieving or reducing agents described herein encompass additional therapies and treatment regimens with other agents in some embodiments. Such additional therapies and treatment regimens can include another pain relieving or reducing therapy in some embodiments. Alternatively, in other embodiments, additional therapies and treatment regimens include other agents used to treat adjunct conditions associated with the cancer or a side effect from such agent in the combination therapy. In further embodiments, adjuvants or enhancers are administered with a combination therapy described herein. Additional pain relieving or reducing therapies include physical therapy, acupunctural therapy, non-pharmacological herbal treatments, or other therapies that are capable of relieving or reducing bone cancer pain in a patient.

### EXAMPLES

### Example 1

### Preparation of the Exemplary Cyclohexenone Compounds

One hundred grams of mycelia, fruiting bodies or mixture of both from Antrodia camphorata were placed into a flask. A proper amount of water and alcohol (70-100% alcohol solution) was added into the flask and were stirred at 20-25° C. for at least 1 hour. The solution was filtered through a filter and 0.45 μm membrane and the filtrate was collected as the extract.

The filtrate of Antrodia camphorata was subjected to High Performance Liquid chromatography (HPLC) analysis. The separation was performed on a RP18 column, the mobile phase consisted of methanol (A) and 0.3% acetic acid (B), with the gradient conditions of 0-10 min in 95%-20% B, 10-20 min in 20%-10% B, 20-35 min in 10%-10% B, 35-40 min in 10%-95% B, at the flow rate of 1 ml/min. The column effluent was monitored with a UV-visible detector.

The fractions collected at 21.2 to 21.4 min were collected and concentrated to yield compound 5, a product of pale yellow liquid. Compound 5 was analyzed to be 4-hydroxy-5-(11-hydroxy-3,7,11-trimethyldodeca-2,6-dienyl)-2,3-dimethoxy-6-methylcyclohex-2-enone with molecular weight of 408 (Molecular formula: C₂₄ H₄₀O₅). ¹H-NMR (CDCl₃) δ (ppm)=1.21, 1.36, 1.67, 1.71, 1.75, 1.94, 2.03, 2.07, 2.22, 2.25, 3.68, 4.05, 5.71 and 5.56. ¹³C-NMR (CDCl₃)δ(ppm): 12.31, 16.1, 16.12, 17.67, 25.67, 26.44, 26.74, 27.00, 30.10, 40.27, 43.34, 59.22, 60.59, 71.8, 120.97, 123.84, 124.30, 131.32, 134.61, 135.92, 138.05, 160.45, and 197.11.

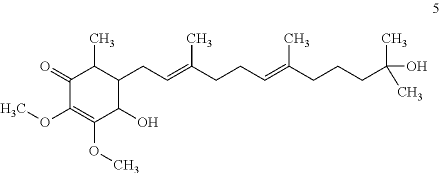 [SMILES: COC1=C(OC)C(O)C(C/C=C(\C)CC/C=C(\C)CCCC(C)(C)O)C(C)C1=O]

### Compound 5: 4-hydroxy-5-(11-hydroxy-3,7,11-trimethyldodeca-2,6-dienyl)-2,3-dimethoxy-6-methylcyclohex-2-enone

The fractions collected at 23.7 to 24.0 min were collected and concentrated to yield compound 7, a product of pale yellow liquid. Compound 7 was analyzed to be 4-hydroxy-2,3-dimethoxy-5-(11-methoxy-3,7,11-trimethyldodeca-2,6-dienyl)-6-methylcyclohex-2-enone with molecular weight of 422 (C₂₅H₄₂O₅). ¹H-NMR (CDCl₃) δ (ppm)=1.21, 1.36, 1.71, 1.75, 1.94, 2.03, 2.07, 2.22, 2.25, 3.24, 3.68, 4.05, 5.12, 5.50, and 5.61. ¹³C-NMR (CDCl₃)δ(ppm): 12.31, 16.1, 16.12, 17.67, 24.44, 26.44, 26.74, 27.00, 37.81, 39.81, 40.27, 43.34, 49.00, 59.22, 60.59, 120.97, 123.84, 124.30, 135.92, 138.05, 160.45 and 197.12.

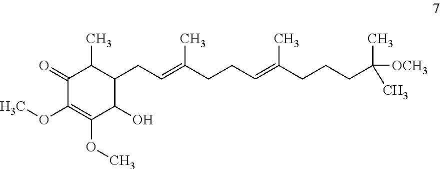 [SMILES: COC1=C(OC)C(O)C(C/C=C(\C)CC/C=C(\C)CCCC(C)(C)OC)C(C)C1=O]

### Compound 7: 4-hydroxy-2,3-dimethoxy-5-(11-methoxy-3,7,11-trimethyldodeca-2,6-dienyl)-6-methylcyclohex-2-enone

The fractions collected at 25 to 30 min were collected and concentrated to yield 4-hydroxy-2,3-dimethoxy-6-methyl-5-(3,7,11-trimethyldodeca-2,6,10-trienyl)cyclohex-2-enone (compound 1), a product of pale yellow brown liquid. The analysis of compound 1 showed the molecular formula of C ₂₄H ₃₈O₄, molecular weight of 390 with melting point of 48 to 52° C. NMR spectra showed that ¹H-NMR (CDCl₃) δ (ppm)=1.51, 1.67, 1.71, 1.75, 1.94, 2.03, 2.07, 2.22, 2.25, 3.68, 4.05, 5.07, and 5.14; ¹³C-NMR (CDCl₃) δ (ppm)=12.31, 16.1, 16.12, 17.67, 25.67, 26.44, 26.74, 27.00, 39.71, 39.81, 40.27, 43.34, 59.22, 60.59, 120.97, 123.84, 124.30, 131.32, 135.35, 135.92, 138.05, 160.45, and 197.12.

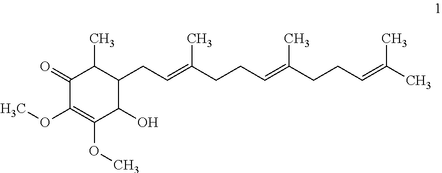 [SMILES: COC1=C(OC)C(O)C(C/C=C(\C)CC/C=C(\C)CCC=C(C)C)C(C)C1=O]

### Compound 1: 4-hydroxy-2,3-dimethoxy-6-methyl-5-(3,7,11-trimethyldodeca-2,6,10-trienyl)cyclohex-2-enone

Compound 6, a metabolite of compound 1, was obtained from urine samples of rats fed with Compound 1 in the animal study. Compound 6 was determined to be 4-hydroxy-2,3-dimethoxy-6-methyl-5-(3-methyl-2-hexenoic acid)cyclohex-2-enone with molecular weight of 312 (C,₆ H₂₄O₆). Compound 4 which was determined as 3,4-dihydroxy-2-methoxy-6-methyl-5-(3,7,11-trimethyldodeca-2,6,10-trienyl)cyclohex-2-enone (molecular weight of 376, C₂₃H₃₆O₄), was obtained when compound 1 was under the condition of above 40° C. for 6 hours.

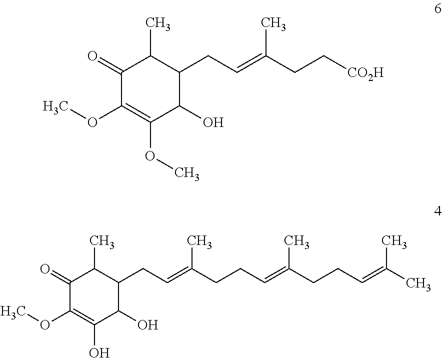 [SMILES: COC1=C(OC)C(O)C(C/C=C(\C)CCC(=O)O)C(C)C1=O ; COC1=C(O)C(O)C(C/C=C(\C)CC/C=C(\C)CCC=C(C)C)C(C)C1=O]

Alternatively, the exemplary compounds may be prepared from 4-hydroxy-2,3-dimethoxy-6-methylcyclohexa-2,5-dienone, or the like.

Similarly, other cyclohexenone compounds having the structure

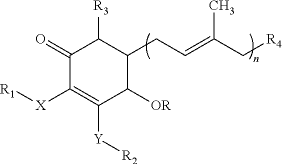

are isolated from Antrodia camphorate or prepared synthetically or semi-synthetically from the suitable starting materials. An ordinary skilled in the art would readily utilize appropriate conditions for such synthesis.

### Example 2

### Effects of Compound 1 in a Rat Model of Bone Cancer Pain

The objective of this study was to assess the potential anti-nociceptive and anti-tumourigenic effects of Compound 1, at doses of 15, 30 and 45 mg/kg, in an animal model of bone cancer pain. Metastasis of cancer cells to the bone was modeled by injecting Walker 256 rat mammary gland carcinoma cells into the medullary cavity of the right tibia (Mao-Yinga, et al. A rat model of bone cancer pain induced by intra-tibia inoculation of Walker 256 mammary gland carcinoma cells. Biochem Biophys Res Commun 2006; 345: 1292-1298). The development of mechanical allodynia was monitored using an established behavioural test (Von Frey test). Treatment administration was chronic, from the day of surgery, and administered twice daily for 21 days to determine whether there was a prophylactic effect on the development of mechanical allodynia. Zoledronic acid was used as a reference substance. No regulatory test guidelines were applicable to this study.

### Test Substance and Materials

The test and reference substances were stored at room temperature.
Test substance: Compound 1
Vehicle for test substance: Corn oil (batch number 058K0070; expiry date 18 Mar. 2014; clear yellow to yellow-green liquid; Sigma, UK)
Reference substance: Zoledronic acid (batch number 50244; expiry date 30 Jun. 2013; clear liquid; prescription medicine from Lyndsay & Gilmour; manufactured by Novartis)

### The Choice of Species, Route of Administration and Dose Levels

Rats have been studied in this model of bone cancer pain. The route of administration of Compound 1 and vehicle was oral. The doses of Compound 1 were 15, 30 and 45 mg/kg, twice a day (approximately 10 h apart) for 21 days.

The dose of zoledronic acid was 30 μg/kg, as a single administration, every second day from the day of surgery, based on historical data. The route of administration of zoledronic acid was subcutaneous.

### Animals

Each animal was arbitrarily allocated a unique identification number which appeared on the data sheets and cage cards. Animals were identified by a waterproof tail mark.
Species: Rat
Strain: Sprague-Dawley
Sex: Female
Number of animals: 60 animals were allocated to study; the remaining 5 animals were returned to stock
Age range: 9 to 12 weeks (based on the average body weight)
Weight range: 181 to 233 g (on day of surgery)
Acclimatisation: 3 days after delivery, before commencing behavioural testing
Source: Harlan UK Ltd

### Location of Study, Housing and Environment

Animals were initially housed in a stock room within the animal house, until transferred to the procedure room. Animals were housed in groups of up to 5 in sawdust filled solid-bottom cages. During the acclimatisation, the rooms and cages were cleaned at regular intervals to maintain hygiene. The rooms were illuminated by fluorescent lights set to give a 12 h light-dark cycle (on 07.00, off 19.00), as recommended in the Home Office Animals (Scientific Procedures) Act 1986. The rooms were air-conditioned and the air temperature and relative humidity measured. During the acclimatisation period room temperature was maintained (range 19 C to 20 C) and humidity levels were within the range 36% to 43%. During the study period temperature was maintained (range 20 C to 21 C) and humidity levels were within the range 27% to 50%.

### Diet and Water

An expanded rodent diet of RM1(E) SQC (Special Diets Services, Witham, UK) and mains tap water were offered ad libitum. Each batch of diet was delivered with an accompanying certificate of analysis (C of A) detailing nutritional composition and levels of specified contaminants (e.g. heavy metals, aflatoxin and insecticides). The water was periodically analysed by The City of Edinburgh Council Analytical and Scientific Services for impurities and contaminants. The criteria for acceptable levels of contaminants in stock diet and water supply were within the analytical specifications established by the diet manufacturer and water analytical service, respectively.

### Health Status

The animals were examined on arrival and prior to the study; all animals were healthy and considered suitable for experimental use.

### Formulation of the Test and Reference Substances

The test substance, Compound 1, was formulated for dosing by dissolving the Compound 1 extract in corn oil to provide concentrations of 3, 6 and 9 mg/mL. No correction factor was applied. The formulations were stored at approximately 4° C. and protected from light until use. The formulated compound was used within 8 days of preparation.

Zoledronic acid is supplied as a pre-formulated solution suitable for injection. A known amount of stock zoledronic acid was diluted using 0.9% w/v sodium chloride to provide a final concentration of 30 μg/mL. No correction factor was applied. A solution was prepared, stored refrigerated, protected from light and used within 8 days of preparation.

A C of A and a material safety data sheet were received with the test substance.

### Group Sizes, Doses and Identification Numbers

There were 5 treatment groups, with up to 12 rats per group. Each treatment group was given a letter (A to E). The rats were randomly allocated to treatment groups on the day of surgery, prior to dosing:

| | | |
|---|---|---|
| C | Vehicle for Compound 1 | 5 mL/kg |
| D | Compound 1 | 15 mg/kg |
| B | Compound 1 | 30 mg/kg |
| A | Compound 1 | 45 mg/kg |
| E | Zoledronic acid | 30 μg/kg |

The dose volume for test substance and vehicle treatments was 5 mL/kg. The vehicle for Compound 1 was corn oil. Each rat allocated to test substance or vehicle treatments received an oral dose, by gavage, twice daily (approximately 8 am and 6 pm) for 21 consecutive days. The dose volume for the reference substance treatment was 1 mL/kg. Each rat allocated to reference substance treatment received a single subcutaneous dose, by injection (approximately 8 am) every second day from the day of surgery.

### Treatment Blinding

Dosing solutions were encoded so that the observer was not aware of the identity of the treatment groups. Due to the nature of the dosing regimen, it was not possible to blind the reference substance formulation to the personnel conducting the dosing procedure. Therefore, this formulation was encoded E.

### Body Weights

Animals were weighed prior to surgery and once on each day of dosing prior to administration of substances, and body weights recorded.

### Daily Observations

General observations were made on all animals on a daily basis from Day 0 PO onwards, with particular attention being paid to the condition of the animal's affected limb.

### Procedure

Cell preparation. Walker 256 rat mammary gland carcinoma cells (obtained from the American Type Culture Collection (ATCC)) were harvested from sub-confluent cultures growing in vitro and the number of viable cells determined. Cells were then re-suspended in sterile phosphate buffered saline (PBS) at a concentration of 4×10⁵ cells. Female Sprague-Dawley rats were intratibially injected in the right leg with 4×10⁵ Walker 256 rat mammary gland carcinoma cells in a volume of 6 μL as detailed in Surgical procedure below.

Acclimatization. Prior to behavioural testing, animals were subjected to routine handling and acclimatisation to the behavioural testing environment.

Baseline behavioural testing. The rats were moved to the procedure room 5 days prior to behavioural testing. The rats were then housed, dosed and observed in the procedure room. The behavioural test was performed on all rats on 2 separate occasions prior to surgery, to establish baseline values. Pre-surgery baseline values were taken as the data from the final (second) day of testing (the data from the first day of testing was not included but classed as part of the acclimatisation).

Mechanical allodynia (Von Frey test): Each animal was placed in a wire mesh cage and a series of Von Frey filaments were applied to the plantar surface of the hind paw, from below. The filaments were applied in ascending order (starting with the weakest force), and the withdrawal threshold for both the left and right hind paws were evaluated. Each filament was indented on the mid-plantar surface of the foot to the point where it just started to bend; this was repeated approximately 8 to 10 times per filament at a frequency of approximately 1 Hz. The withdrawal threshold was defined as the lowest force of two or more consecutive Von Frey filaments to elicit a reflex withdrawal response (i.e. a brief paw flick).

Surgical procedure. The animals were surgically prepared over 2 days. Each rat was anaesthetised as necessary with isofluorane in 1% to 3% oxygen. The surface around the incision site was shaved and sterilised. Under aseptic conditions, an incision was made in the skin over the top of the right tibia to expose the tibia head with minimal damage. Using a needle the tibia was pierced just below the knee joint; this was removed and replaced with a different needle attached to a 10 μL microinjection syringe and the cancer cells (4×10⁵ in 6 μL PBS) were injected into the right intramedullary tibia cavity. The syringe was left in place for approximately 2 min to prevent the carcinoma cells from leaking out of the injection site. The injection site was sealed with bone wax. The overlying muscle and skin was closed using appropriate suture material and the anaesthesia discontinued. On recovery from anaesthesia, rats were re-housed with their cage-mates, on soft padded bedding overnight to reduce the risk of infection, and subsequently on vet bed for approximately one week and then on sawdust bedding following full recovery. The animals were allowed to recover for 5 days before the behavioural testing was recommenced.

Dosing and behavioural testing. The animals were not fasted for this study. Administration of substances was conducted prior to surgery (Day 0), for 21 consecutive days (every second day for the reference substance) up to Day 21 PO. On each day of dosing, the allocated animals each received an oral dose of test substance or vehicle (at approximately 8 am and 6 pm) or a single subcutaneous dose of reference substance (at approximately 8 am on the appropriate days). On Days 6, 12, 14, 19 and 21 PO, the left and right limb of each rat was assessed for mechanical allodynia using the Von Frey test, to investigate treatment effect.

Terminations and tissue collection. Any animal not allocated to a treatment group was returned to stock. During the dosing period, 3 animals (rats 20, 25 and 32) were terminated following a dosing error, 2 animals were terminated on the basis of poor and subdued condition (rats 6 and 13) and 1 animal (rat 18) was terminated and excluded from the study due to the growth of a large tumour at the site of injection.

Allocated animals were euthanized through a rising concentration of carbon dioxide. The right tibia was collected from each animal allocated to the study still remaining on the last day of behavioural testing. Tissue was fixed and stored in 10% formalin. The samples were decalcified, dehydrated and embedded in paraffin before being sectioned on the microtome and stained using haematoxylin and eosin stain. The bones then underwent histological analysis by the Responsible Scientist, to examine the extent of bone destruction and inflammatory cell infiltration across each of the treatment groups.

Statistical Analysis. The Von Frey data were logarithmically transformed (log¹⁰ (force in grams×10 000)) prior to analysis. Statistical comparisons were made between treatment groups using parametric or non-parametric statistical procedures. The choice of parametric or non-parametric test was based on whether the groups to be compared satisfied the homogeneity of variance criterion (evaluated by the Levene Mean test). The reference substance data were analyzed using an unpaired, Student's t-test, with the exception of data from Day 21 left paw (assessed by the F-test) which was analyzed using the Mann-Whitney U-test. Statistical significance was assumed when P<0.05.

### Results

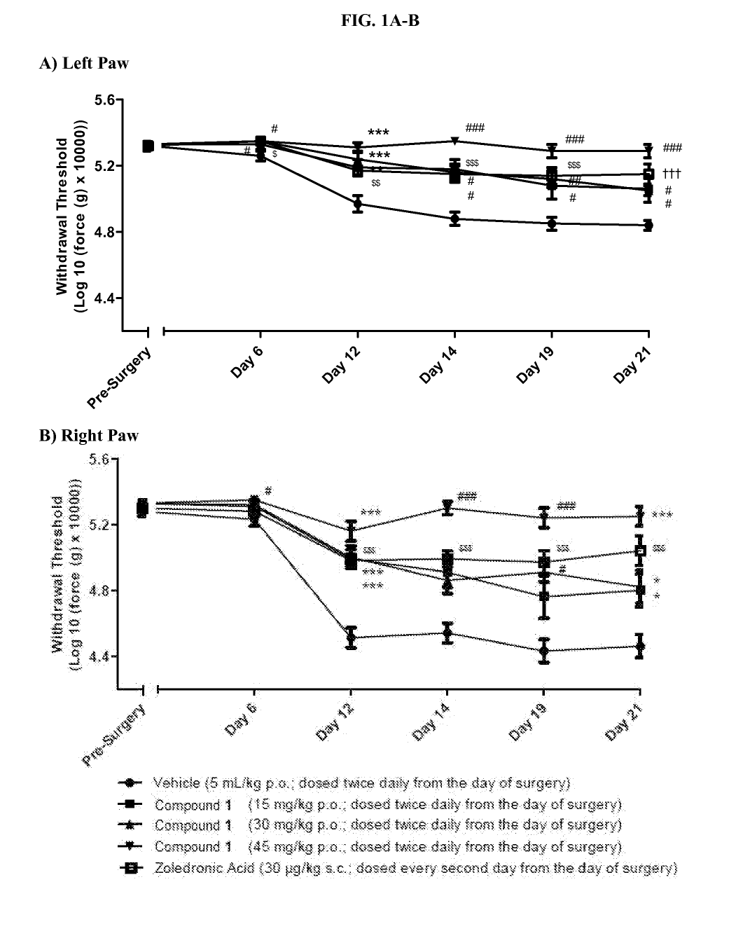

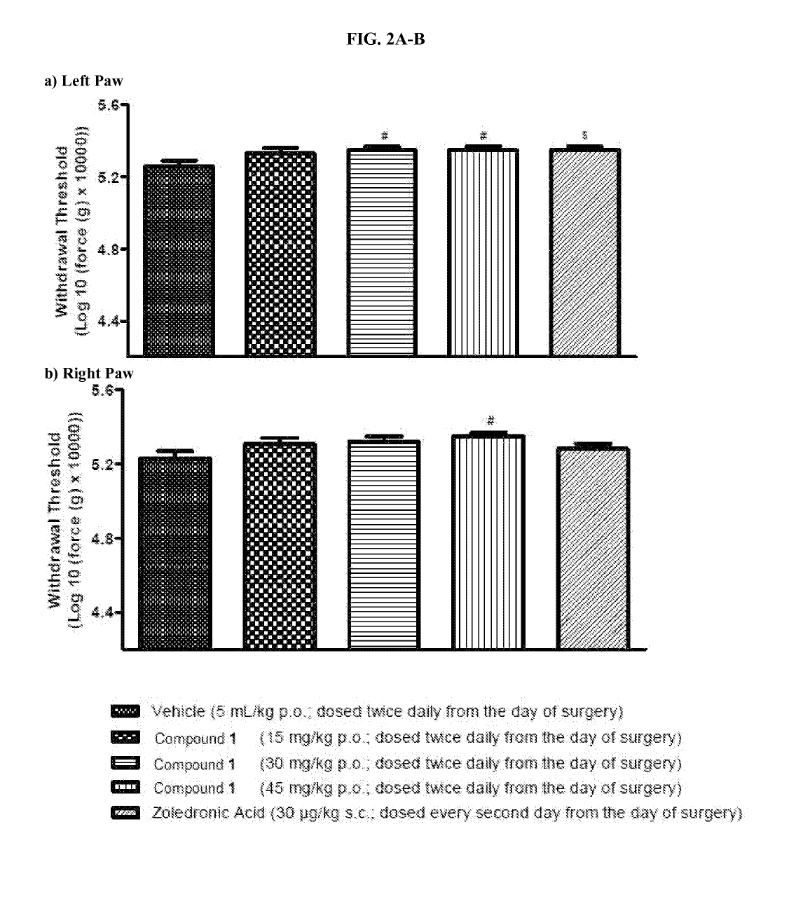

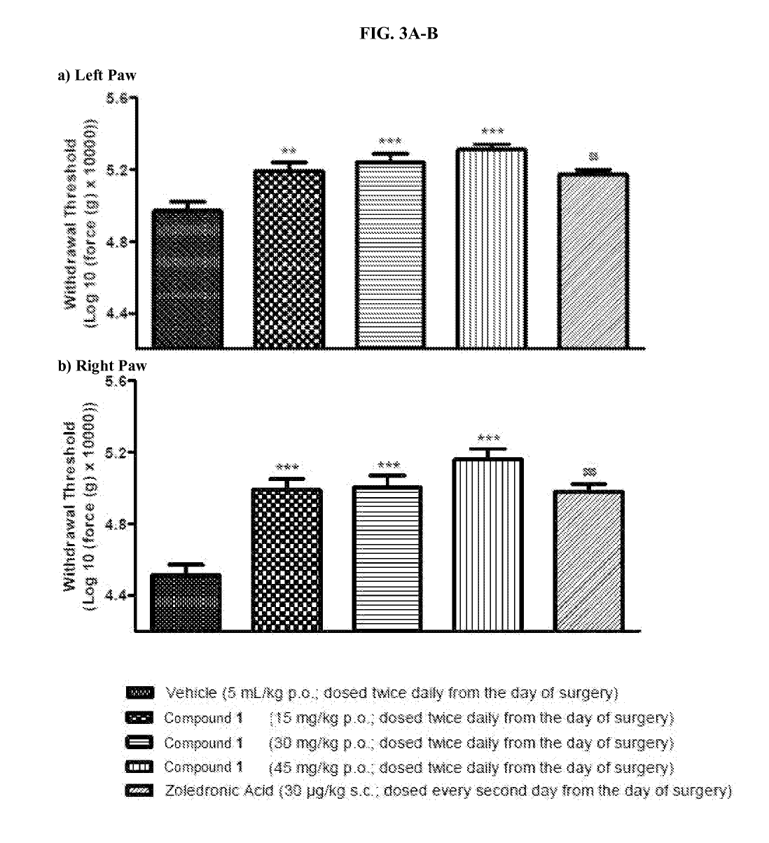

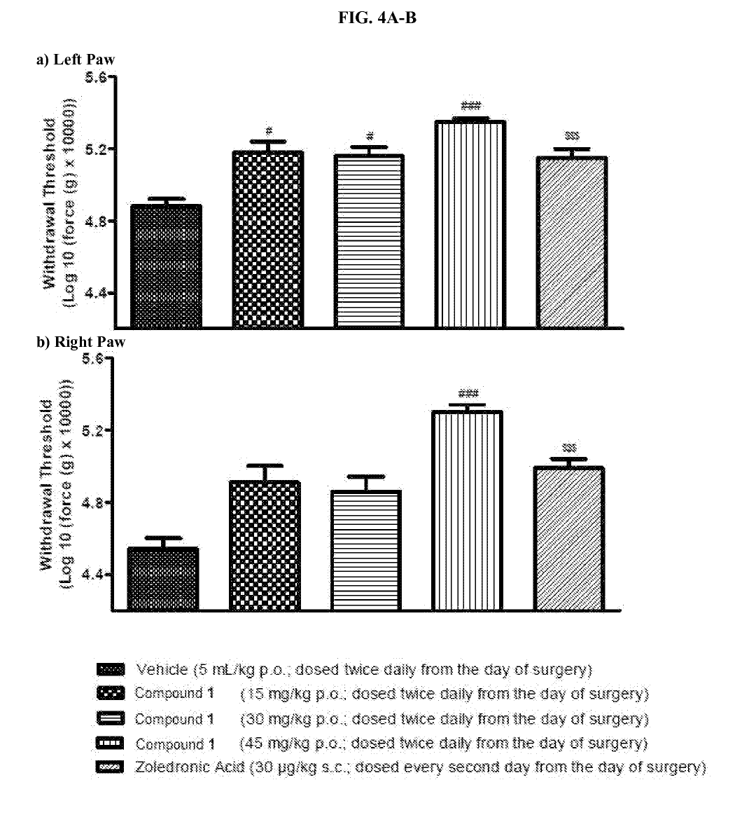

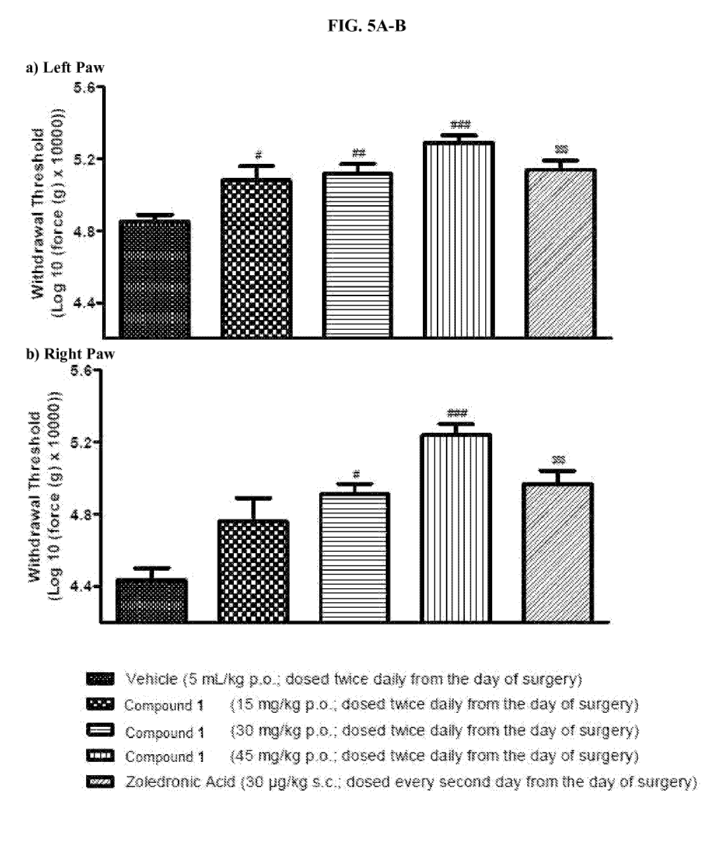

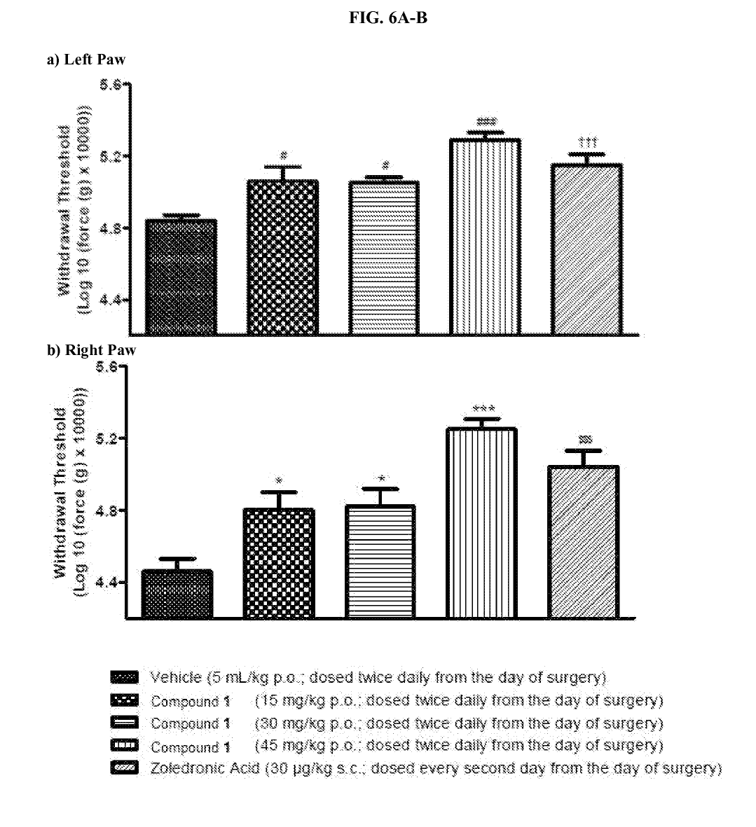

The group mean ±s.e. mean data for the withdrawal threshold is summarized in Table 1 and Table 2 and FIGS. 1-6.

TABLE 1
Effects of Compound 1 on mechanical allodynia
(grams data) in a rat model of bone cancer pain

| | Withdrawal threshold (g) on day post-operative | | | | | |
|---|---|---|---|---|---|---|
| | Pre-Surgery | | Day 6 | | Day 12 | |
| Treatment | L | R | L | R | L | R |
| Vehicle | 21.02 ± | 19.49 ± | 18.73 ± | 17.59 ± | 10.05 ± | 3.57 ± |
| (5 mL/ | 1.20 | 1.37 | 1.39 | 1.50 | 1.48 | 0.48 |
| kg, p.o.) | | | | | (11) | (11) |
| Compound 1 | 21.79 ± | 21.79 ± | 21.65 ± | 20.81 ± | 16.27 ± | 11.12 ± |
| (15 mg/ | 1.03 | 1.03 | 1.12 | 1.29 | 1.77 | 1.95 |
| kg, p.o.) | | | (11) | (11) | (11) | (11) |
| Compound 1 | 21.79 ± | 21.79 ± | 22.55 ± | 21.02 ± | 18.34 ± | 11.15 ± |
| (30 mg/ | 1.03 | 1.03 | 0.77 | 1.20 | 1.78 | 1.61 |
| kg, p.o.) | | | | | (11) | (11) |
| Compound 1 | 21.79 ± | 21.79 ± | 22.55 ± | 22.55 ± | 20.81 ± | 15.98 ± |
| (45 mg/ | 1.03 | 1.03 | 0.77 | 0.77 | 1.29 | 2.17 |
| kg, p.o.) | | | | | (11) | (11) |
| Zoledronic | 21.02 ± | 20.26 ± | 22.55 ± | 19.49 ± | 15.14 ± | 9.95 ± |
| acid | 1.20 | 1.31 | 0.77 | 1.37 | 1.22 | 1.24 |
| (30 μg/ | | | | | | |
| kg, s.c.) | | | | | | |
| | Withdrawal threshold (g) on day post-operative | | | | | |
| | Day 14 | | Day 19 | | Day 21 | |
| Treatment | L | R | L | R | L | R |
| Vehicle | 7.97 ± | 3.81 ± | 7.18 ± | 3.07 ± | 6.99 ± | 3.24 ± |
| (5 mL/ | 0.83 | 0.51 | 0.56 | 0.55 | 0.50 | 0.47 |
| kg, p.o.) | (11) | (11) | (10) | (10) | (10) | (10) |
| Compound 1 | 16.61 ± | 9.97 ± | 13.83 ± | 8.36 ± | 13.74 ± | 7.82 ± |
| (15 mg/ | 2.08 | 1.85 | 2.32 | 2.04 | 2.42 | 1.43 |
| kg, p.o.) | (11) | (11) | (11) | (11) | (11) | (11) |
| Compound 1 | 15.71 ± | 8.55 ± | 14.04 ± | 9.07 ± | 11.40 ± | 8.15 ± |
| (30 mg/ | 1.92 | 1.34 | 1.55 | 1.18 | 0.81 | 1.43 |
| kg, p.o.) | (11) | (11) | (11) | (11) | (11) | (11) |
| Compound 1 | 22.48 ± | 20.41 ± | 20.12 ± | 18.58 ± | 20.12 ± | 19.06 ± |
| (45 mg/ | 0.84 | 1.54 | 1.67 | 2.02 | 1.67 | 2.17 |
| kg, p.o.) | (11) | (11) | (10) | (10) | (10) | (10) |
| Zoledronic | 15.02 ± | 10.28 ± | 14.66 ± | 10.37 ± | 15.46 ± | 13.25 ± |
| acid | 1.61 | 0.96 | 1.68 | 1.48 | 2.06 | 2.28 |
| (30 μg/ | | | | | | |
| kg, s.c.) | | | | | | |

Data are expressed as mean ± s.e. mean.
Vehicle was corn oil.
n = 12 animals per group except where detailed in the parenthesis.
Statistical analysis was conducted on the Log transformed data.

TABLE 2
Effects of Compound 1 on mechanical allodynia
(log data) in a rat model of bone cancer pain

| | Withdrawal Threshold (Log 10 (force (g) × 10 000)) on Day | | | | | |
|---|---|---|---|---|---|---|
| | Post-Operative | | | | | |
| | Pre-Surgery | | Day 6 | | Day 12 | |
| Treatment | L | R | L | R | L | R |
| Vehicle | 5.32 ± | 5.28 ± | 5.26 ± | 5.23 ± | 4.97 ± | 4.51 ± |
| (5 mL/ | 0.03 | 0.03 | 0.03 | 0.04 | 0.05 | 0.06 |
| kg, p.o.) | | | | | (11) | (11) |
| Compound 1 | 5.33 ± | 5.33 ± | 5.33 ± | 5.31 ± | 5.19 ± | 4.99 ± |
| (15 mg/ | 0.02 | 0.02 | 0.03 | 0.03 | 0.05 | 0.06 |
| kg, p.o.) | | | (11) | (11) | (11) ** | (11) *** |
| Compound 1 | 5.33 ± | 5.33 ± | 5.35 ± | 5.32 ± | 5.24 ± | 5.00 ± |
| (30 mg/ | 0.02 | 0.02 | 0.02 # | 0.03 | 0.05 | 0.07 |
| kg, p.o.) | | | | | (11) *** | (11) *** |
| Compound 1 | 5.33 ± | 5.33 ± | 5.35 ± | 5.35 ± | 5.31 ± | 5.16 ± |
| (45 mg/ | 0.02 | 0.02 | 0.02 # | 0.02 # | 0.03 | 0.06 |
| kg, p.o.) | | | | | (11) *** | (11) *** |
| Zoledronic | 5.32 ± | 5.30 ± | 5.35 ± | 5.28 ± | 5.17 ± | 4.98 ± |
| Acid | 0.03 | 0.03 | 0.02 ^{$} | 0.03 | 0.03 ^{$$} | 0.04 ^{$$$} |
| (30 μg/ | | | | | | |
| kg, s.c.) | | | | | | |

| | Withdrawal Threshold (Log 10 (force (g) × 10 000)) on Day | | | | | |
|---|---|---|---|---|---|---|
| | Post-Operative | | | | | |
| | Day 14 | | Day 19 | | Day 21 | |
| Treatment | L | R | L | R | L | R |
| Vehicle | 4.88 ± | 4.54 ± | 4.85 ± | 4.43 ± | 4.84 ± | 4.46 ± |
| (5 mL/ | 0.04 | 0.06 | 0.04 | 0.07 | 0.03 | 0.07 |
| kg, p.o.) | (11) | (11) | (10) | (10) | (10) | (10) |
| Compound 1 | 5.18 ± | 4.91 ± | 5.08 ± | 4.76 ± | 5.06 ± | 4.80 ± |
| (15 mg/ | 0.06 | 0.09 | 0.08 | 0.13 | 0.08 | 0.10 |
| kg, p.o.) | (11) # | (11) | (11) # | (11) | (11) # | (11) * |
| Compound 1 | 5.16 ± | 4.86 ± | 5.12 ± | 4.91 ± | 5.05 ± | 4.82 ± |
| (30 mg/ | 0.05 | 0.08 | 0.05 | 0.06 | 0.03 | 0.10 |
| kg, p.o.) | (11) # | (11) | (11) ## | (11) # | (11) # | (11) * |
| Compound 1 | 5.35 ± | 5.30 ± | 5.29 ± | 5.24 ± | 5.29 ± | 5.25 ± |
| (45 mg/ | 0.02 | 0.04 | 0.04 | 0.06 | 0.04 | 0.06 |
| kg, p.o.) | (11) ### | (11) ### | (10) ### | (10) ### | (10) ### | (10) *** |
| Zoledronic | 5.15 ± | 4.99 ± | 5.14 ± | 4.97 ± | 5.15 ± | 5.04 ± |
| Acid | 0.05 ^{$$$} | 0.05 ^{$$$} | 0.05 ^{$$$} | 0.07 ^{$$$} | 0.06 ^{†††} | 0.09 ^{$$$} |
| (30 μg/ | | | | | | |
| kg, s.c.) | | | | | | |

Data is expressed as mean ± s.e. mean.
Vehicle was corn oil.
n = 12 animals per group except where detailed in the parenthesis.
* P < 0.05,
** P < 0.01 and
*** P < 0.001 when compared to vehicle (ANOVA and Dunnett's test).
# P < 0.05,
## P < 0.01 and
### P < 0.001 when compared to vehicle (Kruskall Wallis and Dunn's test).
^{$} P < 0.05,
^{$$} P < 0.01 and
^{$$$} P < 0.001 when compared to vehicle (unpaired, Student's t-test).
^{†††} P < 0.001 when compared to vehicle (Mann Whitney U-test).

### Development of Mechanical Allodynia

The development of mechanical allodynia following an intratibial injection of Walker 256 cells in to the right leg was investigated using an established behavioural test, namely Von Frey filaments. Mechanical allodynia was evident in the vehicle control group where the animals exhibited a marked increase in sensitivity of the right hind paw to the Von Frey filaments as early as Day 6 PO, indicative of the tumour development and physiological changes associated with metastasis of the bone. There was also a notable increase in the sensitivity of the left hind paw to the Von Frey filaments over the duration of the study, indicative of the phenomenon of ‘mirror image pain’. The mechanisms behind this are not fully understood, but are thought to be centrally acting.

### Effects of Compound 1 on the Development of Mechanical Allodynia

Twice daily oral administration of Compound 1 (from the day of surgery) at doses of 30 and 45 mg/kg had significant protective effects from as early as Day 6 PO. By Day 12, all Compound 1 treatment groups were significantly less sensitive in the left and right paws to the Von Frey filaments than the vehicle control group and this continued for the duration of the study. By Day 21 PO, the right hind paw withdrawal threshold was significantly less sensitive following oral administration of Compound 1 at doses of 15 mg/kg (7.82 ±1.43 g; P<0.05; ANOVA and Dunnett's test), 30 mg/kg (8.15±1.43 g; P<0.05; ANOVA and Dunnett's test) and 45 mg/kg (19.06±2.17 g; P<0.001; ANOVA and Dunnett's test) when compared to the vehicle group data (3.24±0.47 g). Similarly, by Day 21 PO, the left hind paw withdrawal threshold was significantly less sensitive following oral administration of Compound 1 at doses of 15 mg/kg (13.74±2.42 g; P<0.05; Kruskal Wallis and Dunn's test), 30 mg/kg (11.40±0.81 g; P<0.05; Kruskal Wallis and Dunn's test) and 45 mg/kg (20.12±1.67 g; P<0.001; Kruskal Wallis and Dunn's test) when compared to the vehicle group data (6.99±0.50 g). These data indicate a dose-dependent increase in the withdrawal threshold in response to the Compound 1 administration, with the high dose treatment group demonstrating magnitude of double that observed at the lower dose levels. The withdrawal threshold recorded for the high dose treatment group on Day 21 shows a reversal of the sensitivity of both paws to levels similar to the pre-surgery baseline.

### Effects of Zoledronic Acid on the Development of Mechanical Allodynia

Subcutaneous administration of zoledronic acid (every second day from the day of surgery) at a dose of 30 μg/kg had significant protective effects from as early as Day 6 PO (left paw). By Day 12, the withdrawal thresholds for the reference animals were significantly less sensitive in the left and right paws to the Von Frey filaments than the vehicle group and this continued for the duration of the study. By Day 21 PO, the right hind paw withdrawal threshold was significantly increased (13.25±2.28 g; P<0.001; unpaired, Student's t-test) when compared to the vehicle group data (3.24±0.47 g) and the left hind paw withdrawal threshold was significantly increased (15.46±2.06 g; P<0.001; Mann Whitney U-test) when compared to the vehicle group data (6.99±0.50 g). These data are consistent with that reported in the literature.

### Conclusion

Oral administration of Compound 1 at doses of 15, 30 and 45 mg/kg (twice daily from the day of surgery for 21 days) had a significant prophylactic effect against the establishment of mechanical allodynia in this model. The effects observed were evident from as early as Day 6 PO, and over the course of the study increased in magnitude, as the level of allodynia developed in the vehicle control group. Both the affected and contralateral hind limbs were protected by the Compound 1 treatment. The withdrawal thresholds across the time course of the study observed in the high dose Compound 1 treatment group were consistent with pre-surgery baseline values, indicating that this dose level was highly effective in the prevention of tumour formation and subsequent establishment of mechanical allodynia. These data indicate that Compound 1 is effective in the prevention of bone cancer pain in the clinic.

Subcutaneous administration of zoledronic acid (every second day from the day of surgery) at a dose of 30 μg/kg had significant protective effects from as early as Day 6 PO (left paw). By Day 12, the withdrawal thresholds for the reference animals were significantly less sensitive in the left and right paws to the Von Frey filaments than the vehicle group and this continued for the duration of the study. This is consistent with the known pharmacological properties of zoledronic acid as a bisphosphonate compound, used in the treatment of bone cancer.

### Example 3

### Efficacy of Compound 1 in the Treatment of Bone Metastases-related Pain

This study will evaluate the efficacy and safety of 50 mg Compound 1 administered intravenously every second day in the treatment of bone metastases-related pain in patients with prostate cancer.
Study Type: Interventional
Study Design: Allocation: Non-Randomized
   Endpoint Classification: Safety/Efficacy Study
   Intervention Model: Single Group Assignment
   Masking: Open Label
   Primary Purpose: Treatment

### Primary Outcome Measures:

To measure the intensity of the pain relief of the patients at the end of treatment with a five classes score (TOTPAR=TOTal PAin Relief) [Time Frame: at 12 weeks or at 16 weeks (end of treatment)]

### Secondary Outcome Measures:

To measure the intensity of the pain relief of the patients with the PAR at each visit [Time Frame: every 3 or 4 weeks during 12 to 16 weeks] [Designated as safety issue: Yes]

To evaluate the pain variation with VAS between V1 and V2, V3, V4, V5. [Time Frame: every 3 or 4 weeks during 12 to 16 weeks] [Designated as safety issue: Yes]

To evaluate the pain variation with BPI (=Brief Pain Inventory) and correlate with VAS (=Visual Analog Scale) [Time Frame: every 3 or 4 weeks during 12 to 16 weeks] [Designated as safety issue: Yes]

To evaluate the use of analgesic (analgesic score) and the number of patients needing an analgesic radiotherapy between V1 and V5 [Time Frame: every 3 or 4 weeks during 12 to 16 weeks] [Designated as safety issue: Yes]

To evaluate the duration of responses [Time Frame: at 12 weeks or at 16 weeks (end of treatment)] [Designated as safety issue: Yes]

To evaluate the number of skeletal related events by patient [Time Frame: every 3 or 4 weeks during 12 to 16 weeks] [Designated as safety issue: Yes]

To evaluate the effect on functional disability, professional activity (BPI), the PS and overall condition (VAS) between V1 and V5 [Time Frame: every 3 or 4 weeks during 12 to 16 weeks] [Designated as safety issue: Yes]

To evaluate the variations of PSA (=Prostate specific Antigen) between V1 and End of study or premature withdrawal [Time Frame: at 12 weeks or at 16 weeks (end of treatment)] [Designated as safety issue: Yes]

### Eligibility

Ages Eligible for Study: 18 Years and older (60 to 100 people); Genders Eligible for Study: Male; Accepts Healthy Volunteers: No.

### Criteria

Inclusion Criteria:
Histologically proven adenocarcinoma of the prostate
Bone-scan documented metastases
Age>18 years
Non-controlled bone pain despite systemic anti-tumor therapy (hormone or chemotherapy) initiated at least 4 weeks before inclusion
Life expectancy>3 months
Written informed consent

Exclusion Criteria:
New systemic anti-tumor therapy initiated less than 4 weeks before study entry or predictable need for starting a new treatment within 8 weeks
Radiation therapy on bone target lesions or bone-targeted isotope therapy (strontium or samarium) completed less than 4 weeks before study entry
Bisphosphonate therapy within 8 weeks before study entry
Abnormal renal function (serum creatinine >2×the upper normal limit or creatinine clearance <30 ml/min)
Corrected serum calcium>3 mmol/L or <2 mmol/L
Clinically relevant hypersensitivity to zoledronic acid, or another bisphosphonate, or one component present in the formulation of the study drug
Severe concomitant medical condition that could hamper patient's quality of life or influence the interpretation of pain
Patients unable to fill in a questionnaire (neurologic or psychiatric conditions, illiteracy, etc.)
Other protocol-defined exclusion criteria may apply.

### Example 4

### Parenteral Formulation

To prepare a parenteral pharmaceutical composition suitable for administration by injection, 100 mg of a compound or its salt described herein is dissolved in DMSO and then mixed with 10 mL of 0.9% sterile saline. The mixture is incorporated into a dosage unit form suitable for administration by injection.

### Example 5

### Oral Formulation

To prepare a pharmaceutical composition for oral delivery, 100 mg of an exemplary Compound 1 was mixed with 100 mg of corn oil. The mixture was incorporated into an oral dosage unit in a capsule, which is suitable for oral administration.

In some instances, 100 mg of a compound described herein is mixed with 750 mg of starch. The mixture is incorporated into an oral dosage unit for, such as a hard gelatin capsule, which is suitable for oral administration.

### Example 6

### Sublingual (Hard Lozenge) Formulation

To prepare a pharmaceutical composition for buccal delivery, such as a hard lozenge, mix 100 mg of a compound described herein, with 420 mg of powdered sugar mixed, with 1.6 mL of light corn syrup, 2.4 mL distilled water, and 0.42 mL mint extract. The mixture is gently blended and poured into a mold to form a lozenge suitable for buccal administration.

### Example 7

### Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

### Example 8

### Rectal Gel Formulation

To prepare a pharmaceutical composition for rectal delivery, 100 mg of a compound described herein is mixed with 2.5 g of methylcelluose (1500 mPa), 100 mg of methylparapen, 5 g of glycerin and 100 mL of purified water. The resulting gel mixture is then incorporated into rectal delivery units, such as syringes, which are suitable for rectal administration.

### Example 9

### Topical Gel Composition

To prepare a pharmaceutical topical gel composition, 100 mg of a compound described herein is mixed with 1.75 g of hydroxypropyl cellulose, 10 mL of propylene glycol, 10 mL of isopropyl myristate and 100 mL of purified alcohol USP. The resulting gel mixture is then incorporated into containers, such as tubes, which are suitable for topical administration.

### Example 10

### Ophthalmic Solution Composition

To prepare a pharmaceutical ophthalmic solution composition, 100 mg of a compound described herein is mixed with 0.9 g of NaCl in 100 mL of purified water and filtered using a 0.2 micron filter. The resulting isotonic solution is then incorporated into ophthalmic delivery units, such as eye drop containers, which are suitable for ophthalmic administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method for treating, reducing, or managing bone cancer pain comprising administering to a subject a therapeutically effective amount of a compound having the structure: 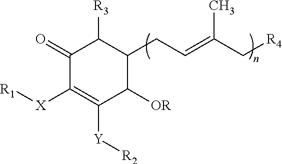
wherein each of X and Y independently is oxygen, NR₅ or sulfur;
R is a hydrogen or C(═O)C₁-C₈alkyl;
each of R₁, R₂ and R₃ independently is a hydrogen, methyl or (CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl, wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m=1-12; and
n=1-12; or a pharmaceutically acceptable salt, metabolite, solvate or prodrug thereof.

2. The method of claim 1, which further comprises administering to the patient a therapeutically or prophylactically effective amount of at least one second active agent.

3. The method of claim 2, wherein the second active agent is capable of relieving or reducing pain.

4. The method of claim 1, wherein the bone cancer pain is from cancer originated in bone.

5. The method of claim 1, wherein the bone cancer pain is from osteosarcoma.

6. The method of claim 1, wherein the bone cancer pain is from cancer metastasized to bone.

7. The method of claim 6, wherein the bone cancer pain is from breast cancer, prostate cancer, lung cancer, renal cancer, liver cancer, kidney cancer, bladder cancer, thyroid cancer, cervical cancer, or colon cancer metastasized to bone.

8. The method of claim 6, wherein the bone cancer pain is from esophageal cancer, or nasopharyngeal cancer metastasized to bone.

9. The method of claim 6, wherein the bone cancer pain is from sarcoma metastasized to bone.

10. The method of claim 7, wherein the bone cancer pain is from breast cancer, prostate cancer, renal cancer, or lung cancer, metastasized to bone.

11. The method of claim 2, wherein the at least one second active agent is selected from the group consisting of an antidepressant, antihypertensive, anxiolytic, calcium channel blocker, muscle relaxant, non-narcotic analgesic, anti-inflammatory agent, cox-2 inhibitor, alpha-adrenergic receptor agonist, alpha-adrenergic receptor antagonist, ketamine, anesthetic, immunomodulatory agent, immunosuppressive agent, corticosteroid, hyperbaric oxygen, anticonvulsant, and a combination thereof.

12. The method of claim 2, wherein the at least one second active agent is selected from the group consisting of salicylic acid acetate, celecoxib, ketamine, gabapentin, carbamazepine, oxcarbazepine, phenytoin, sodium valproate, prednisone, nifedipine, clonidine, oxycodone, meperidine, morphine sulfate, hydromorphone, fentanyl, acetaminophen, ibuprofen, naproxen sodium, griseofulvin, amitriptyline, imipramine, doxepin, and combinations thereof

13. The method of claim 1, wherein said compound is isolated from Antrodia camphorate.

14. The method of claim 1, wherein R is a hydrogen, C(═O)C₃H₈, C(═O)C₂H₅, or C(═O)CH₃.

15. The method of claim 1, wherein each of R₁, R₂ and R₃ independently is a hydrogen, methyl, ethyl, propyl, butyl, pentyl, hexyl, heptyl, or octyl.

16. The method of claim 15, wherein R₁ or R₂ is a hydrogen or methyl.

17. The method of claim 1, wherein R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein the 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

18. The method of claim 1, wherein R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

19. The method of claim 18, wherein R₄ is CH₂CH═C(CH₃)₂.

20. The method of claim 19, wherein said compound is 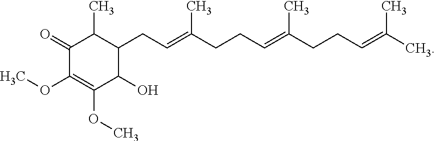 [SMILES: COC1=C(OC)C(O)C(C/C=C(\C)CC/C=C(\C)CCC=C(C)C)C(C)C1=O]
